(19) United States
(12) Patent Application Publication
(10) Pub. No.: US 2014/0227236 A1
(43) Pub. Date: Aug. 14, 2014

(54) HIV-RESISTANT STEM CELLS AND USES THEREOF

(71) Applicant: StemCyte, Inc., Ewing, NJ (US)

(72) Inventor: Wise Young, New Brunswick, NJ (US)

(73) Assignee: StemCyte, Inc., Ewing, NJ (US)

(21) Appl. No.: 14/256,215

(22) Filed: Apr. 18, 2014

Related U.S. Application Data
(63) Continuation of application No. 13/263,431, filed on Oct. 7, 2011.
(60) Provisional application No. 61/167,967, filed on Apr. 9, 2009.

(51) Int. Cl. A61K 35/48

(52) U.S. Cl. CPC A61K 35/51; A61K 2035/124
USPC 424/93.21; 435/372

(57) ABSTRACT

Disclosed are recombinant stem cells that are resistant to HIV infection. Also disclosed are their uses in treating AIDS.

### RELATED APPLICATION

This application claims priority to U.S. Provisional Application No. 61/167,967, filed on Apr. 9, 2009, the content of which is hereby incorporated by reference in its entirety.

### BACKGROUND

Human Immunodeficiency Virus (HIV-1) causes Acquired Immunodeficiency Syndrome (AIDS) in both adults and children. It was estimated that HIV infected about 370,000 children in 2007 alone. Of about two million deaths from AIDS in 2007, one of seven was a child. Indeed, mother-to-child transmission of HIV accounts for a vast majority of the children infected with HIV. While such transmission can be prevented with anti-viral therapies of the mothers and careful limitation of exposure to maternal fluids during delivery, many babies continue to be infected for various reasons. First, current treatments of HIV-infected babies are limited and relatively ineffective. Second, AIDS tends to progress rapidly in babies. Third, special diagnostic techniques are in need for detecting the virus in babies of less than 18 months old as they may not develop antibodies to HIV or they may have antibodies derived from the mothers. Finally, although anti-retroviral therapies can be used to treat the babies, these therapies do not restore immune functions that are essential for children to survive common childhood illnesses, such as chickenpox and mumps. Thus, there is a need for methods for treating AIDS in babies.

### SUMMARY

This invention is based on, at least in part, unexpected discoveries that stem cells, such as umbilical cord blood cells collected at birth of a infant, can be transfected to make them resistant to HIV-1 infections, and that transfusing the transfected cells back to the infant or another human subject produces HIV-resistant blood and immune cells. Thus, the transfected cells can be used for treating AIDS without causing myeloablation.

Accordingly, one aspect of this invention features a method for treating a human subject having, or at risk of having, an HIV infection. The method includes obtaining human stem cells containing a first RNAi agent that represses the expression of CCR5 and a second RNAi agent that represses the expression of CXCR4 and administering to a human subject in need thereof an effective amount of the stem cells. CCR5 and CXCR4 are chemokine receptors, which are essential for HIV infection of lymphocytes and macrophages. These modified stem cells are resistant to HIV infection and can form, both in vitro and in vivo, colony-forming units (CFU) and engraft and restore immune function in immune deficient human subjects (e.g., babies). In one example, the stem cells are stem cells found in human umbilical cord blood cells.

The above described method can be used to treat a baby born of a mother that has an HIV infection. Preferably, the stem cells (e.g., umbilical cord blood cells) are autologous to the subject. The umbilical cord blood cells can be obtained by a process including transiently transferring into the cells (1) the first RNAi agent or a first nucleic acid encoding the first RNAi agent and (2) the second RNAi agent or a second nucleic acid encoding the second RNAi agent. The preparation process can further include introducing into the cells a recombinant nucleic acid encoding a selectable marker protein, and enriching the cells expressing the selectable marker protein. The above-mentioned cells can further contain a third RNAi agent that represses the expression of another gene that is essential for HIV reproduction or infection. Examples of there genes include those encoding CD4, HIV-1 gag, HIV-1 vive, HIV-1 tat, and HIV-1 rev. In one embodiment, a non-viral method is used to transfect umbilical cord blood (neonatal blood) with short inhibiting RNAs (siRNA) that block the synthesis of chemokine receptors (such as CCR5 and CXCR4).

Another aspect of this invention features an isolated human stem cell (e.g., umbilical cord blood cell) or a composition containing such cells. The cell contains the above-discussed first RNAi agent and a second RNAi agent.

A subject to be treated can be identified by standard diagnosing techniques for an HIV infection. “Treating” refers to administration of a composition (e.g., a cell composition) to a subject, who is suffering from or is at risk for developing that disorder, with the purpose to cure, alleviate, relieve, remedy, delay the onset of, or ameliorate the disorder, the symptom of the disorder, the disease state secondary to the disorder, or the predisposition toward the damage/disorder. An “effective amount” refers to an amount of the composition that is capable of producing a medically desirable result in a treated subject. The treatment method can be performed alone or in conjunction with other drugs or therapies. A subject refers to a human or a non-human animal. Examples of a non-human animal include all vertebrates, e.g., mammals, such as non-human primates (particularly higher primates). In a preferred embodiment, the subject is a human. In another embodiment, the subject is an experimental animal or animal suitable as a disease model.

The details of one or more embodiments of the invention are set forth in the description below. Other features, objects, and advantages of the invention will be apparent from the description, the drawings, and the claims. All references cited herein are to aid in the understanding of the invention, and are incorporated in their entireties for all purposes without limitation.

### BRIEF DESCRIPTION OF DRAWINGS

FIG. 1 is a drawing showing chemokine co-receptors. HIV-1 binds to CD4 and CCR5 co-receptor present on activated T-cells. Natural ligands of CCR5 include RANTES, MIP-1a, and MIP-1b, preventing HIV-1 binding to CCR5. HIV-1 also binds to CXCR4. SDF-1 is the natural ligand for CXCR4 and forces the receptor to internalize and to be less availability as a co-receptor for HIV binding. While CCR5 is a co-receptor with CD4 for HIV-1 binding, CXCR4, CCR3, and CCR2b can bind HIV-1 without CD4. HIV-1 rarely binds to CCR1 but may do so with other co-receptors. The receptors are G-protein coupled and activation of CCR5 receptors stimulates viral replication. Blockade of the G-proteins tends to reduce viral replication.

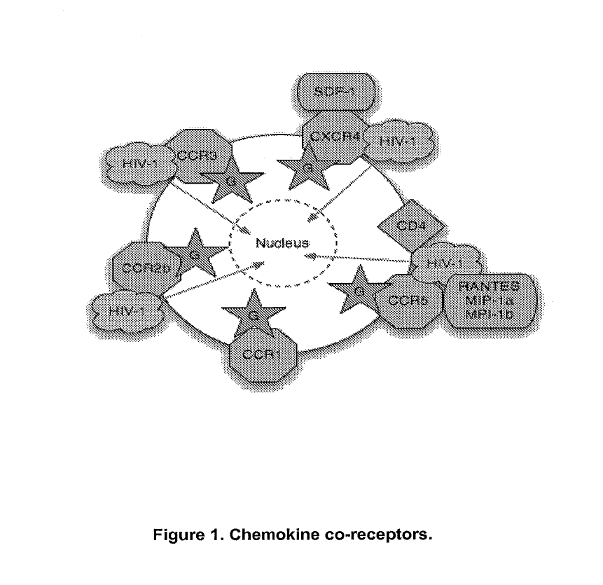

FIG. 2 is a drawing showing non-genetic methods of reducing HIV-1 infection. Administration of RANTES as part of an IgG fusion protein markedly reduces HIV-1 binding to CCR5 and consequent infection of cells. Interferon-beta reduces expression of both CD4 and CCR5 while increasing the production of RANTES, preventing infection. Autoantibodies or vaccine-induced antibodies against CXCR4 may reduce CXCR4 expression on macrophages and other immune cells.

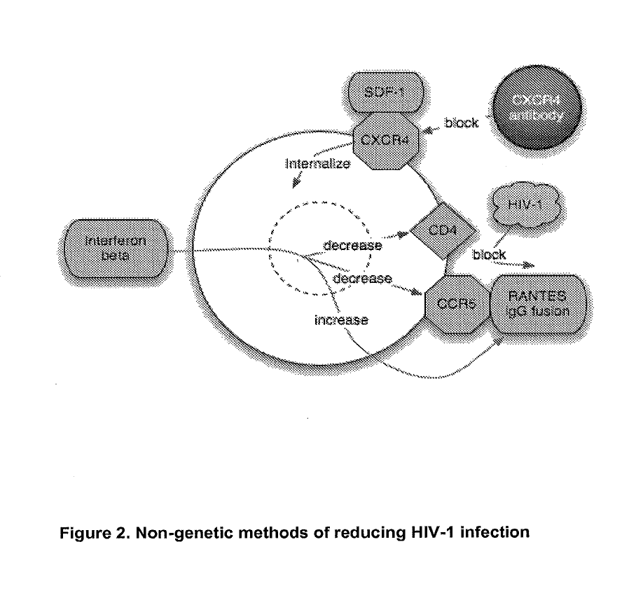

FIG. 3 is a drawing showing chemokine receptor suppression methods. Several approaches have been devised to suppress CXCR4 and CCR5 co-receptor expression on the surface of cells. The most efficient and popular method is RNA interference (RNAi) methods to prevent transcription of the receptor proteins. Another approach is to use ribozymes that break down specific RNA. The RANTES Kdel method attaches an endoplasmic reticulum sequence to RANTES. This anchors the RANTES in the endoplasmic reticulum where it can trap the CCR5 protein.

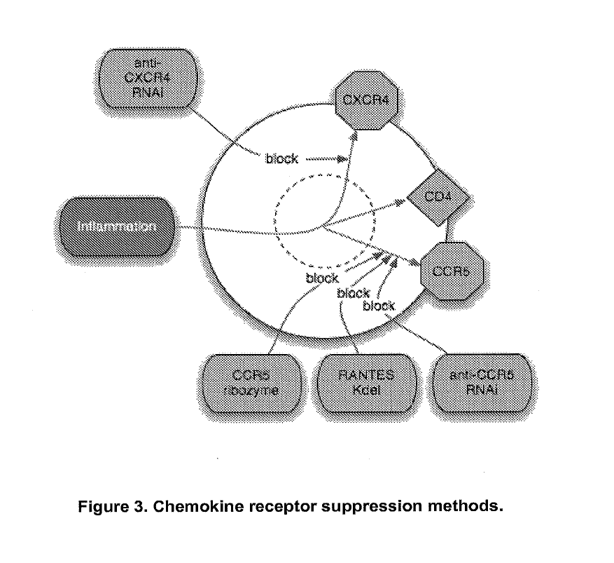

FIG. 4 is a drawing showing combination therapy of AIDS. Several therapies are indicated in green boxes. For example, the TAR decoy sets up a decoy in the nucleoli to attract tat RNA, a critical component of HIV-1 envelope protein. The anti-tat siRNA breaks down the tat RNA. The anti-rev siRNA breaks down the rev RNA, another important envelope protein. The anti-CCR5 siRNA or ribozyme are methods for increasing breakdown of the CCR5 RNA and therefore its expression. Blocking fusogenic envelope glycoprotein proteins gp41 and gp120 also prevent infection, with drugs such as T20 (enfurvirtide) and C34. Antibody against CD4 (anti-CD mab), maraviroc, and other drugs can a also block the receptors.

FIG. 5 is a drawing showing a proposed umbilical cord blood treatment. Cord blood mononuclear cells are isolated with Ficoll gradient after osmotic shock to remove red blood cells and platelets, in the presence DNAase. Four genes are transduced into the cells by electroporation: CCR5Δ32, neomycin resistance gene, green fluorescence protein (GFP), and CXCR4 siRNA. The CCR5Δ32 is a mutated form of the CCR5 that binds to CCR5 and prevents it from reaching the surface. CXCR4 siRNA is a short inhibitory RNA that prevents the transcription of CXCR4. Green fluorescent allows successfully transfected cells to be observed. The neomycin resistance gene allows the cell to be resist neomycin toxicity, simplifying the purification of the transfected cells.

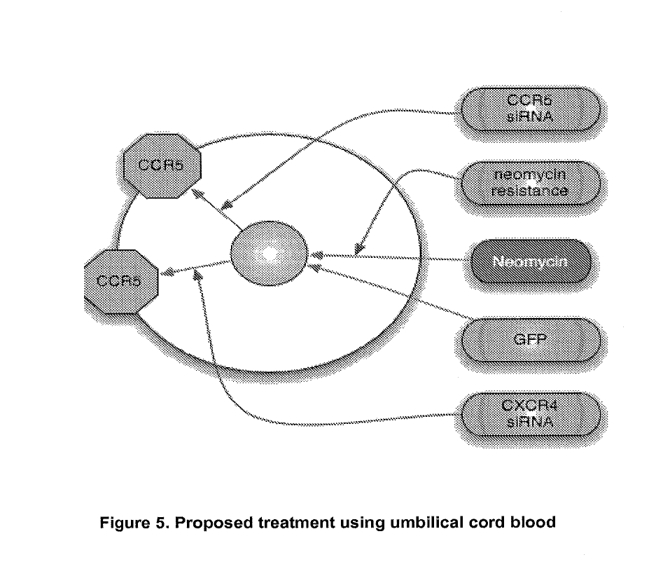

### DETAILED DESCRIPTION

This invention relates to treating AIDS using stem cells that are resistant to HIV infection.

### Stem Cells

Various stem cells can be used in this invention. Examples of the stem cells include umbilical cord blood cells, hematopoietic stem cells, embryonic stem cells, and other stem cells that can differentiate into functional immune cells, such as T-helper cells.

The term “stem cell” refers to a cell that is capable of differentiating into a number of final, differentiated cell types. Stem cells may be totipotent or pluripotent. Totipotent stem cells typically have the capacity to develop into any cell type. Totipotent stem cells can be both embryonic and non-embryonic in origin. Pluripotent cells are typically cells capable of differentiating into several different, final differentiated cell types. Unipotent stem cells can produce only one cell type, but have the property of self-renewal which distinguishes them from non-stem cells. These stem cells can originate from various tissue or organ systems, including, but not limited to, blood, nerve, muscle, skin, gut, bone, kidney, liver, pancreas, thymus, and the like. In accordance with the present invention, the stem cell can be derived from an adult or neonatal tissue or organ.

The cells described in this invention are substantially pure. The term “substantially pure”, when used in reference to stem cells or cells derived therefrom (e.g., differentiated cells), means that the specified cells constitute a substantial portion of or the majority of cells in the preparation (i.e., more than 20%, 30%, 40%, 50%, 60%, 70%, 80%, 90%, or 95%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%).

In a preferred embodiment, umbilical cord blood cells are used. These stem cells can be enriched by methods known in the art and then tested by standard techniques. To confirm the differentiation potential of the cells, they can be induced to form, for example, various colony forming units, by methods known in the art.

The cells thus confirmed can be further propagated in a non-differentiating medium culture for more than 10, 20, 50, or 100 population doublings without indications of spontaneous differentiation, senescence, morphological changes, increased growth rate, or changes in ability to differentiate into neurons. The cells can be stored by standard methods before use.

The terms “proliferation” and “expansion” as used interchangeably herein with reference to cells, refer to an increase in the number of cells of the same type by division. The term “differentiation” refers to a developmental process whereby cells become specialized for a particular function, for example, where cells acquire one or more morphological characteristics and/or functions different from that of the initial cell type. The term “differentiation” includes both lineage commitment and terminal differentiation processes. Differentiation may be assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the stem cells. For example, neural progenitor cells and muscle progenitor cells can differentiate into hematopoietic cell lineages. The terms “lineage commitment” and “specification,” as used interchangeably herein, refer to the process a stem cell undergoes in which the stem cell gives rise to a progenitor cell committed to forming a particular limited range of differentiated cell types. Committed progenitor cells are often capable of self-renewal or cell division. The term “terminal differentiation” refers to the final differentiation of a cell into a mature, fully differentiated cell. For example, neural progenitor cells and muscle progenitor cells can differentiate into hematopoietic cell lineages, terminal differentiation of which leads to mature blood cells of a specific cell type. Usually, terminal differentiation is associated with-withdrawal from the cell cycle and cessation of proliferation. The term “progenitor cell,” as used herein, refers to a cell that is committed to a particular cell lineage and which gives rise to cells of this lineage by a series of cell divisions. An example of a progenitor cell would be a myoblast, which is capable of differentiation to only one type of cell, but is itself not fully mature or fully differentiated.

### RNAi/Nucleic Acid/Vector

The above-described stem cells can be transfected to express one or more RNAi agents (e.g., RNAi agents against CCR5 or CXCR4) that render the cells resistant to HIV.

The term “RNAi” or “RNA interference” refers to a sequence-specific or selective process by which a target molecule (e.g., a target gene, protein or RNA) is down-regulated. Within the scope of this invention is utilization of RNAi featuring degradation of RNA molecules (e.g., within a cell). Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free double-stranded RNA, which directs the degradative mechanism. Alternatively, RNAi can be initiated by the hand of man, for example, to silence the expression of target genes.

The term “RNAi agent” refers to an RNA (or analog thereof), having sufficient sequence complementarity to a target RNA (i.e., the RNA being degraded) to direct RNAi. A RNA agent having a “sequence sufficiently complementary to a target RNA sequence to direct RNAi” means that the RNA agent has a sequence sufficient to trigger the destruction of the target RNA by the RNAi machinery (e.g., the RISC complex) or process. A RNA agent having a “sequence sufficiently complementary to a target RNA sequence to direct RNAi” also means that the RNA agent has a sequence sufficient to trigger the translational inhibition of the target RNA by the RNAi machinery or process. A RNA agent can also have a sequence sufficiently complementary to a target RNA encoded by the target DNA sequence such that the target DNA sequence is chromatically silenced. In other words, the RNA agent has a sequence sufficient to induce transcriptional gene silencing, e.g., to down-modulate gene expression at or near the target DNA sequence, e.g., by inducing chromatin structural changes at or near the target DNA sequence. The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

Small, interfering RNA (siRNA) molecules are typically double stranded RNA molecules (RNA is usually single stranded) which inhibit expression of its target mRNA. As used herein, the term siRNA may include what is sometimes referred to as short hairpin RNA (shRNA) molecules. Typically, shRNA molecules consist of short complementary sequences separated by a small loop sequence wherein one of the sequences is complimentary to the gene target. shRNA molecules are typically processed into an siRNA within the cell by endonucleases.

RNAi sequences encoded by the RNAi expression cassettes of the present invention result in the expression of small interfering RNAs that are short, double-stranded RNAs that are not toxic in normal mammalian cells. There is no particular limitation in the length of such DNA derived RNAi (ddRNAi) agents as long as they do not show cellular toxicity. RNAis can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

The termini of a ddRNAi agent according to the present invention may be blunt or cohesive (overhanging) as long as the ddRNAi agent effectively silences the target gene. The cohesive (overhanging) end structure is not limited only to a 3′ overhang, but a 5′ overhanging structure may be included as long as the resulting ddRNAi agent is capable of inducing the RNAi effect. In addition, the number of overhanging nucleotides may be any number as long as the resulting ddRNAi agent is capable of inducing the RNAi effect. For example, if present, the overhang may consist of 1 to 8 nucleotides; preferably it consists of 2 to 4 nucleotides.

The ddRNAi agent utilized in the present invention may have a stem-loop structured precursor (shRNA) in which the ends of the double-stranded RNA are connected by a single-stranded, linker RNA. The length of the loop portion of the shRNA may be 5 to 20 bp in length, and is preferably 5 to 9 bp in length

The nucleic acid sequences that are targets for the RNAi expression cassettes of the present invention include genes that are involved in HIV reproduction or infection. The sequences for the RNAi agent or agents are selected based upon the genetic sequence of the target gene sequence(s); and preferably are based on regions of the target gene sequences that are conserved. Methods of alignment of sequences for comparison and RNAi sequence selection are well known in the art. The determination of percent identity between two or more sequences can be accomplished using a mathematical algorithm. Preferred, non-limiting examples of such mathematical algorithms are the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (http://www.ncbi.nlm.nih.gov/).

Typically, inhibition of target sequences by RNAi requires a high degree of sequence homology between the target sequence and the sense strand of the RNAi molecules. In some embodiments, such homology is higher than about 70%, and may be higher than about 75%. Preferably, homology is higher than about 80%, and is higher than 85% or even 90%. More preferably, sequence homology between the target sequence and the sense strand of the RNAi is higher than about 90%, 91%, 92%, 93%, 94%, 95%, 96%, 97%, 98% or 99%.

In addition to selecting the RNAi sequences based on conserved regions of a target gene, selection of the RNAi sequences may be based on other factors. Examples of the factors include percent GC content, position from the translation start codon, or sequence similarities based on an in silico sequence database search for homologs of the proposed RNAi, thermodynamic pairing criteria. Alternatively, individual specific candidate RNAi polynucleotide sequences typically can be generated and tested to determine whether interference with expression of a desired target can be elicited.

When using a ddRNAi agent, the RNAi expression cassette is ligated into a delivery vector. The constructs into which the RNAi expression cassette is inserted and used for high efficiency transduction and expression of the ddRNAi agents in various cell types may be derived from viruses and are compatible with viral delivery; alternatively, non-viral delivery method may be used. Generation of the construct can be accomplished using any suitable genetic engineering techniques well known in the art, including the standard techniques of PCR, oligonucleotide synthesis, restriction endonuclease digestion, ligation, transformation, plasmid purification, and DNA sequencing. If the construct is a viral construct, the construct preferably comprises, for example, sequences necessary to package the RNAi expression construct into viral particles and/or sequences that allow integration of the RNAi expression construct into the target cell genome. The viral construct also may contain genes that allow for replication and propagation of virus, though in other embodiments such genes are supplied in trans. Additionally, the viral construct may contain genes or genetic sequences from the genome of any known organism incorporated in native form or modified. For example, a preferred viral construct may comprise sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the RNAi expression cassette, an internal ribosomal entry site (IRES) sequence can be included. Preferably, the additional genetic elements are operably linked with and controlled by an independent promoter/enhancer. In addition a suitable origin of replication for propagation of the construct in bacteria may be employed. The sequence of the origin of replication generally is separated from the ddRNAi agent and other genetic sequences that are to be expressed in the cells. Such origins of replication are known in the art and include the pUC, ColE1, 2-micron or SV40 origins of replication.

Vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502-09). Preferably, RNA polymerase III promoters are employed. Preferable expression vectors for expressing the siRNA molecules of the invention are plasmids and viral vectors (see, e.g., Sui et al. (2002) PNAS 99:5515-5520; Xia et al. (2002) Nature Biotech. 20:1006-1010; Barton and Medzhitov (2002) PNAS 99:14943-14945; Brummelkamp et al. (2002) Science 296:550-553; Devroe and Silver (2002) BMC Biotechnol., 2(1):15; Tiscornia et al. (2003) PNAS, 100:1844-1848).

### Delivery Systems

The RNAi expression constructs and RNAi agents of the present invention may be introduced into the target cells in vitro or ex vivo and then subsequently placed into a patient to affect therapy, or administered directly to a patient by in vivo administration. Target cells can be obtained from cord blood, bone marrow, peripheral blood or any other method for obtaining stem cells known in the art.

Virus-Based System

A viral delivery system based on any appropriate virus may be used to deliver the RNAi expression constructs of the present invention. In addition, hybrid viral systems may be of use. The choice of viral delivery system depends on various parameters, such as efficiency of delivery into cells, transduction efficiency of the system, pathogenicity, immunological and toxicity concerns, and the like. When selecting a viral delivery system to use in the present invention, it is important to choose a system where RNAi expression construct-containing viral particles are preferably: 1) reproducibly and stably propagated; 2) able to be purified to high titers; and 3) able to mediate targeted delivery (delivery of the multiple-promoter RNAi expression construct to the desired cells without widespread dissemination). In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Unlike retrovirus, adenovirus, and herpes simplex virus, AAV appears to lack human pathogenicity and toxicity (Kay, et al., Nature. 424: 251 (2003) and Thomas, et al., Nature Reviews, Genetics 4:346-58 (2003)).

Another viral delivery system useful with the RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

Lentiviruses can also be used in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription from the integrated provirus.

Other viral systems known to those skilled in the art may be used to deliver the RNAi expression cassettes of the present invention to cells. Examples oft hem include gene-deleted adenovirus-transposon vectors that stably maintain virus-encoded transgenes in vivo through integration into host cells (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus;.

Non-Viral Systems

Alternatively, the RNAi expression cassettes or related vectors may be delivered into cells by non-viral means. Examples include calcium phosphate co-precipitation, DEAE-dextran-mediated transfection, lipofection, electroporation, or microinjection. Again, methods not affecting the pluripotency of the cells are preferred. Description of such techniques can be found in, e.g., U.S. Pat. Nos. 7,422,736 and 5,591,625 and US Patent Application NO. 20020127715. Further examples include bacterial vectors or mini-circles (see Chen, et al., Molecular Therapy. 8(3):495-500 (2003) and US Pat. Pub. 2004/0214329). Mini-circles are non-viral DNA vectors that provide for persistently high expression of nucleic acid transcription. Mini-circle vectors are characterized by being devoid of expression-silencing bacterial DNA sequences, and may include a unidirectional site-specific recombination product sequence in addition to a ddRNAi expression cassette.

The above-described nucleic acid or vector can also be delivered by the use of polymeric, biodegradable microparticle or microcapsule delivery devices known in the art. Another way to achieve uptake of the nucleic acid is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al., 1995, J. Mol. Med. 73:479). Alternatively, tissue specific targeting can be achieved by the use of tissue-specific transcriptional regulatory elements that are known in the art. Delivery of “naked DNA” (i.e., without a delivery vehicle) to an intramuscular, intradermal, or subcutaneous site is another means to achieve in vivo expression.

A common transfection reagents are charged lipophilic compounds that are capable of crossing cell membranes. When these are complexed with an RNAi agent they can act to carry the RNAi agent across the cell membrane. A large number of such compounds are available commercially. Polyethylenimine (PEI) is a class of transfection reagents, chemically distinct from lipophilic compounds that act in a similar fashion to lipophilic compounds, but have the advantage they can also cross nuclear membranes. An example of such a reagent is ExGen 500 (Fermentas). A construct according to the present invention may be packaged as a linear fragment within a synthetic liposome or micelle for delivery into the target cell.

Another delivery method useful for the method of this invention comprises the use of Cyclosert™ technology as described in U.S. Pat. No. 6,509,323. This technology platform is based upon cup-shaped cyclic repeating molecules of glucose known as cyclodextrins. The “cup” of the cyclodextrin molecule can form “inclusion complexes” with other molecules, making it possible to combine the CYCLOSERT polymers with other moieties to enhance stability or to add targeting ligands. In addition, cyclodextrins have generally been found to be safe in humans (individual cyclodextrins currently enhance solubility in FDA-approved oral and IV drugs) and can be purchased in pharmaceutical grade on a large scale at low cost. These polymers are extremely water soluble, non-toxic and non-immunogenic at therapeutic doses, even when administered repeatedly. The polymers can easily be adapted to carry a wide range of small-molecule therapeutics at drug loadings that can be significantly higher than liposomes.

Chemically modified siRNA molecules may be employed in the instant invention. Examples of such chemical modifications include, without limitation, phosphorothioate internucleotide linkages, 2′-O-methyl ribonucleotides, 2′-deoxy-2′-fluoro ribonucleotides, 2′-deoxy ribonucleotides, “universal base” nucleotides, 5-C-methyl nucleotides, and inverted deoxyabasic residue incorporation. Preferably, the chemical modifications preserve the inhibition activity of the unmodified siRNA molecule in cells while, at the same time, increasing the serum stability of these compounds or other favorable property of the siRNA molecules. U.S. patent application Publication No. 20050032733, incorporated herein by reference, provides numerous suitable modifications of siRNA molecules.

### Uses and Applications

The stem cells described in this invention can be used in a variety of ways. One can use the cells for treating AIDS in a human subject. In particular, they can be used to treat infants born of mothers having HIV.

For example, one can isolate umbilical cord blood cells from such a new born. After that, one can introduce into the cells an expression nucleic acid vector encoding the above-described RNAi agents in the manner described above. After delivering the vector into the cells, one can transplant the cells back into the newborn using methods known in the art. As the cells are produced from the same person, the treatment does not cause immune rejection.

Alternatively, one can make universal donor cells generated from stem cells (e.g., umbilical cord blood cells) prepared from a healthy subject. The method for making universal donor cells are known in the art and that for making universal donor cells for treating AIDS will be described below.

Under proper conditions, the transplanted cells can develop into functional blood cells and immune cells. To facilitate this development, the patient may be administered with factors to induce the development of the cells. Such factors can be small molecule compounds, peptides, and nucleic acids. Examples include cytokines promoting the differentiation of immune cells.

The above-described cells and methods can be used in various gene therapy methods known in the art. Gene therapy includes both ex vivo and in vivo techniques. Specifically, the above-described stem cells can be genetically engineered ex vivo with an oligonucleotide modulator or a nucleic acid molecule encoding the modulator, with the engineered cells then being provided to a patient to be treated. Cell cultures may be formulated for administration to a patient, for example, by dissociating the cells (e.g., by mechanical dissociation) and intimately admixing the cell with a pharmaceutically acceptable carrier (e.g., phosphate buffered saline solution). Alternatively, cells may be cultured on a suitable biocompatible support and transplanted into a patient. The engineered cells are typically autologous so as to circumvent xenogeneic or allotypic rejection. Such ex vivo methods are well known in the art.

The above-described stem cells can be genetically engineered to generate histocompatible donor cells or tissues for transplantation to other patients. The goal of transplantation and cell therapy is to successfully replace failing tissues or organs with functional donor tissues or organs. However, for transplantation to succeed, two major barriers need to be overcome: the availability of suitable donor tissues or organs and immune rejection. The replacement of failing tissues or organs and the treatment of the rejection is restricted by the limited number of acceptable donors and the need for co-administration of toxic immuno-suppressive drugs in conjunction with long term immuno-suppressive protocols. Current and experimental transplantation protocols rely mainly on sibling donors, other small pools of allogeneic donors, and xenogeneic donors. The above-described genetically engineered stem cells can be used to overcome these limitations.

More specifically, the stem cells described herein can be genetically engineered to not express on their surface class II MHC molecules. More preferably, the cells are engineered to not express substantially all cell surface class I and class II MHC molecules. As used herein, the term “not express” mean either that an insufficient amount is expressed on the surface of the cell to elicit a response or that the protein that is expressed is deficient and therefore does not elicit a response.

The MHC molecules refer to HLA molecules, specifically of classes HLA A, B and C, and class II HLA DP, DQ, and DR, and their subclasses. This terminology is generally construed as specific to the human MHC, but is intended herein to include the equivalent MHC genes from the donor cell species, for example, if the cells are of porcine origin, the term HLA would refer to the equivalent porcine MHC molecules, whether MHC I or II. When the class II MHC molecules are removed, CD4+ T-cells do not recognize the genetically engineered endothelial cells; when both the class I and class II MHC molecules are removed neither CD4+ nor CD8+ cells recognize the modified cells.

The preferred genetic modification performed on the stem cells includes 1) disrupting the endogenous invariant chain gene which functions in the assembly and transport of class II MHC molecules to the cell surface and loading of antigenic peptide, and 2) disrupting the endogenous β₂-microglobulin gene (β₂M gene) which codes for a protein required for the cell surface expression of all class I MHC molecules. Alternatively, just the invariant chain gene is disrupted. Invariant chain is believed to be required for the insertion of antigienic peptide fragments into the MHC class II molecule. Together, the antigenic peptide and MHC is recognized by T cells. In the absence of antigenic peptide, T cell recognition is not normally obtained, nor is the MHC class II molecule folded properly. Thus, in cells lacking invariant chain, presentation of peptide will be abrogated and even if minuscule amounts of cell surface MHC are obtained, they may be devoid of peptide and therefore, non-immunogenic.

Disruption of these genes can be accomplished by means of homologous recombination gene targeting techniques. These techniques are well known in the art. See U.S. Pat. Nos. 6,916,654 and 6,986,887, Zijlstra et al., 1989, Nature 342:435438; and Koller et al., 1990 Science 248:1227-1230.

### Compositions

The present invention provides for pharmaceutical compositions containing the above-descried cells and optionally other active anti-HIV agents/compounds (e.g., drugs for treating AIDS). Examples of anti-HIV agents include HIV vaccines, protease inhibitors (e.g., INDINAVIR, RITONAVIR, SAQINAVIR, NELFINAVIR, and AMPRENAVIR), nucleoside reverse transcriptase inhibitors (e.g., ZIDOVUDINE (AZT), DIDANOSINE, ZALCITABINE, LAMIVUDINE, STAVUDINE, and ABACAVIR), non-nucleoside reverse transcriptase inhibitors (e.g., NEVIRAPINE, DELAVIRDINE, and EFAVIRENZ), integrase inhibitors, and fusion inhibitors.

Pharmaceutical compositions can be prepared by mixing a therapeutically effective amount of the cells and, optionally, other active agents/compounds, with a pharmaceutically acceptable carrier. The carrier can have different forms, depending on the route of administration.

The just-described pharmaceutical compositions can be prepared by conventional pharmaceutical excipients and methods of preparation. All excipients may be mixed with disintegrating agents, solvents, granulating agents, moisturizers, and binders. As used herein, the term “effective amount” or ‘therapeutically effective amount’ refers to an amount which results in measurable amelioration of at least one symptom or parameter of a specific disorder. A therapeutically effective amount of the above-descried cells can be determined by methods known in the art. An effective amount for treating a disorder can easily be determined by empirical methods known to those of ordinary skill in the art. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of the above-described disorders is within the scope of the invention. Clinically significant attenuation or amelioration means perceptible to the patient and/or to the physician.

The phrase “pharmaceutically acceptable” refers to molecular entities and other ingredients of such compositions that are physiologically tolerable and do not typically produce unwanted reactions when administered to a human. Preferably, the term “pharmaceutically acceptable” means approved by a regulatory agency of the federal or a state government or listed in the U.S. Pharmacopeia or other generally recognized pharmacopeia for use in mammals, and more particularly in humans. Pharmaceutically acceptable salts, esters, amides, and prodrugs refers to those salts (e.g., carboxylate salts, amino acid addition salts), esters, amides, and prodrugs which are, within the scope of sound medical judgment, suitable for use in contact with the tissues of patients without undue toxicity, irritation, allergic response, and the like, commensurate with a reasonable benefit/risk ratio, and effective for their intended use.

A carrier applied to the pharmaceutical compositions described above refers to a diluent, excipient, or vehicle with which a compound is administered. Such pharmaceutical carriers can be sterile liquids, such as water and oils. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The above-descried cells or active agents can be administered to individuals through infusion or injection (for example, intravenous, intrathecal, intramuscular, intraluminal, intratracheal, intraperitoneal, or subcutaneous), orally, transdermally, or other methods known in the art. Administration may be once every two weeks, once a week, or more often, but frequency may be decreased during a maintenance phase of the disease or disorder.

Both heterologous and autologous cells can be used. In the former case, HLA-matching should be conducted to avoid or minimize host reactions. In the latter case, autologous cells are enriched and purified from a subject and stored for later use. The cells may be cultured in the presence of host or graft T cells ex vivo and re-introduced into the host. This may have the advantage of the host recognizing the cells as self and better providing reduction in T cell activity.

The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the above-described composition. Dosages and administration regimen can be adjusted depending on the age, sex, physical condition of administered as well as the benefit of the conjugate and side effects in the patient or mammalian subject to be treated and the judgment of the physician, as is appreciated by those skilled in the art. In all of the above-described methods, the cells can be administered to a subject at 1×10⁴ to 1×10¹⁰/time.

### The CKR5Δ32 Mutation

HIV-1 binds to CD4+ monocytes but requires a co-receptor to infect the cells. The chemokine receptor 5 (CKR5 or CCR5) serves a secondary receptor for about half of the strains of HIV-1. A particular mutation of CCR5 confers powerful protection against infection by HIV-1 exposure. Located on human chromosome 3p21, the CCR5 gene is missing a 32-base pair allele called (CKR5Δ32). Present in approximately 10% of Caucasians in Europe and the United States, this mutation confers protection against AIDS. Note that the murine CCR5, even though it is 82% identical to the human CCR5, does not support HIV-1 binding (Atchison, et al., 1996, Science. 274: 1924-6), explaining the resistance of mouse lymphocytes to HIV-1. Patients that are homozygous for CKR5Δ32 remain HIV-1 antibody negative despite repeated exposures to HIV-1. Dean, et al. (Dean et al., 1996, Science. 273: 1856-62) examined 1955 patients who were part of six well-characterized cohort studies of AIDS. Seventeen patients who were CKR5Δ32 homozygotes were amongst 612 people who were exposed to HIV-1 but were HIV-1 antibody negative. Amongst 1343 individuals who were HIV-1 infected, none were homozygote for CKR5Δ32. The frequency of CKR5Δ32 was also significantly greater in individuals who survived HIV-1 infection for more than 10 years. Likewise, Huang, et al. (Huang et al., 1996, Nat Med. 2: 1240-3) found that no CKR5Δ32 homozygote amongst 1252 individuals infected by HIV but 3.6% of HIV-exposed but uninfected participants were CKR5Δ32 homozygotes. The CKR5Δ32 mutation may have evolved as protection against the plague and smallpox (Galvani et al., 2003, Proc Natl Acad Sci USA. 100: 15276-9; Hedrick et al., 2006, Trends Genet 22: 293-6; Sabeti et al., 2005, PLoS Biol. 3: e378; and Stumpf et al., 2004, Trends Ecol Evol. 19: 166-8) that were pandemic in the Europe 500-600 years ago. The mutation was found in 11-14th century remains in Poland with a prevalence of about 5% (Zawicki et al., 2008, Infect Genet Evol. 8: 146-51). Some recent research suggest that the CKR5Δ32 mutation was present as early as the Bronze Age, pushing back the age of the mutation to at least 3000 and possibly 5000 years ago. The bubonic plague is not a likely selecting factor because absent CCR5 increases susceptibility to Yersinia pests (Styer et al., 2007, Microbes Infect. 9: 1135-8). The vaccinia virus, which confers resistance to smallpox, prefers to infect CD8+ t-cells that express CCR5 (Vanpouille et al., 2007. J Virol. 81: 12458-64). The CKR5Δ32 gene probably evolved in Europe. The CKR5Δ32 mutation declines in frequency from 0.13 amongst Caucasian Russians to 0.12, 0.85, 0.06, 0.05, 0.04, 0.03, and 0.00 respectively amongst Tatars, Uzbeks, Kazakhs, Azerbaijanis, Uigurts, Tuvinians, and Georgians. Homozygous individuals with the defective CKR-5 allele do not express detectable CCR5 on their cell surfaces and can survive multiple exposures to HIV-1 infections (Liu et al., 1996, Cell. 86: 367-77). CKR5 heterozygotes occur in 10-20% of European Caucasians and slow disease progression (Huang et al., 1996, Nat Med. 2: 1240-3 and Rowe P M (1996). CKR-5 deletion heterozygotes progress slower to AIDS. Lancet. 348: 947). CKR5Δ32 does not confer absolute protection (Balotta et al., 1997, Aids. 11: F67-71). Macrophages express CCR5. Peripheral macrophages express CCR5 and tuberculosis enhances CCR5 expression and HIV-1 replication in macrophages. Entry of M-tropic but not T-tropic virus is prevented in dendritic cells from individuals who lack a functional CCR5 receptor (Granelli-Piperno et al., 1996, J Exp Med. 184: 2433-8). Likewise, ischemia and endotoxins upregulate CCR5 receptor expression by rat brain microglia (Spleiss et al., 1998, J Neurosci Res. 53: 16-28), suggesting that CCR5 may play a role in the susceptibility of brain macrophages to HIV-1 infection.

### CXCR4 and Other Receptors Also Affect HIV Susceptibility

The chemokine receptors CXCR4, CCR3, and CCR2b also may serve as co-receptors for HIV-1 (Alkhatib et al., 1997, J Biol Chem. 272: 20420-6). Ayehunie, et al. (Ayehunie, et al., 1997, Blood. 90: 1379-86) showed that HIV-1 enters dendritic cells through a variety of CC and CXC chemokine co-receptors. Bjorndal, et al. (Bjorndal et al., 1997, J Virol. 71: 7478-87) used glioma cell lines expressing CCR1, CCR2b, CCR3, CXCR4, and CCR5 to study HIV-1 isolates. Infections by slow/low isolates were restricted to cells expressing CCR5 while rapid/high isolates used multiple chemokine receptors including CCR5, CXCR4, CCR3, and CCR2b. Xu, et al. (Xu et al., 2008, J Infect Dis. 197: 309-18) found that blood monocytes harbor diversified HIV-1 phenotypes that bind to multiple chemokine receptors. All use CCR5 but some use CXCR4, some CCR3, and some use multiple co-receptors (CCR1, CCR3, GPR15, CCR5, CXCR4). The CXCR4 receptor is also called fusin or Lestr (Simmons et al., 1996, J Virol. 70: 8355-60). SDF-1 is the physiological ligand for CXCR. SDF-1 causes rapid internalization of CXCR4 and profoundly inhibits HIV entry into CD4+ lymphocytes. In early stages of HIV infection, viral isolates bind CCR5 while isolates from later stages of HIV infections tend to bind CXCR4 (Bleul et al., 1997, Proc Natl Acad Sci USA. 94: 1925-30). MT-2 (Macrophage Trophic 2) positive HIV-1 strains enter macrophage through CXCR4 (Bract et al., 1997, Aids. 11: 1415-9). Some HIV-1 strains can interact with CXCR4 independent of CD4 (Hesselgesser et al., 1997, Curr Biol. 7: 112-21). HTV-2 strains utilize CXCR4 and, to a lesser extent CCR3, for cell fusion (Bron et al., 1997, J Virol. 71: 8405-15). CXCR4 blockers can also prevent HIV-1 infections. For example, one anti-CXCR4 factor is the macrophage-derived chemokine ligand 22 (CCL22). HIV-1 can bind and enter T cells by binding CCR3 (Aasa-Chapman et al., 2006, J Virol. 80: 10884-9). Certain HIV-1 strains may use CCR3 on macrophages. Th1 and Th2 cells are defined by their cytokine profiles and expression of CCR5 and CCR3. Alkhatib, et al. (Alkhatib et al., 1997, J Biol Chem. 272: 20420-6) showed that CCR3 interacts certain macrophage (M)-tropic HIV-1 strains that use CCR5, T-cell line (T)-tropic HIV-1 strains that use CXCR4, and dual tropic strains. CCR1 is the closest homologue to CCR3 (53% amino acid identity) but CCR1 is not an HIV-1 co-receptor. Like CXCR4, CCR3 may serve as a CD4 independent receptor for HIV-1 infection of brain cells (Martin-Garcia et al., 2006, Virology. 346: 169-79). Initial evidence suggested that CCR2 might be a co-receptor for HIV-1 infections. In 1998, Ksotrikis, et al. (Kostrikis et al., 1998, Nat Med. 4: 350-3) reported that a conservative substitution in the coding region of CCR2 is associated with slower disease progression but not HIV-1 transmission. Since CCR2 is rarely used as a co-receptor by HIV-1 and the mutation is in a transmembrane region, the authors proposed and found that the CCR2-V641 allelle is associated with a point mutation in the CCR5 regulatory region. Hendel, et al. (Hendel et al., 1998, J Acquir Immune Defic Syndr Hum Retrovirol. 19: 381-6) found significant associations of mutant alleles of CCR5 (p<0.04) but not for CCR2 (p=0.09) or SDF1 (p=0.12) in patients with long term slow AIDS progression. Magierowska, et al. (Magierowska et al., 1999, Blood. 93: 936-41) used combined genotypes of CCR5, CCR2, SDF1, and HLA genes to predict long term non-progressive status of HIV-1 infected individuals.

In summary, while most HIV-1 isolates utilize CCR5 as a co-receptor to enter T cells, HIV-1 may enter macrophages, dendritic cells, and brain cells using other co-receptors. CXCR4 and CCR3 may act as co-receptors for HIV-1 but CCR2 does not appear to be a co-receptor for HIV-1. Antibodies against CD40 suppress HIV-1 that does not use CCR5/CXCR4.

### Chemokine Receptor Blockade Prevents HIV Infections

HIV-1 targets CD4+ T cells by binding CD4 and CCR5 or CXCR4. Both CCR5 and CXCR4 are chemokine receptors (De Clercq et al., 2001, Antivir Chem Chemother. 12 Suppl 1: 19-31). Viral entry can be inhibited by natural ligands for CXCR4, the CXC chemokine SDF-1, the chemokines RANTES, MIP-1 alpha, and MIP-1 beta. Several peptides have also been identified as CXCR4 antagonists and show anti-HIV activity, including bicyclam derivatives. AMD3100 is a specific CXCR4 antagonist. TAK-779 is an anti-HIV quarternary ammonium derivative that interacts with CCR5. CD4+ T cells secrete several natural HIV suppressive factors. Anti-CCR5 factors include macrophage inflammatory protein-1 alpha (MIP-1alpha or CCL3), macrophage inflammatory protein-1 beta (MIP-1beta or CCL4), and RANTES (regulated upon activation of normal T-cells expressed and secreted or CCL5). These chemokines not only inhibit infection of CD4+ T cells by primary, non-syncytium-inducing (NSI) HIV-1 strains but also block env-mediated cell-cell membrane fusion. Stimulating CCR5 contributes to viral replication (Rahbar et al., 2006, J Virol. 80: 7245-59) and blocking CCR5 reduces HIV replication (Arenzana-Seisdedos et al., 1996, Nature. 383: 400).

HIV entry inhibition drugs are particularly important for patients who develop HIV-1 infections that become resistant to combination anti-viral therapies (2006, GMHC Treat Issues. 20: 4-7). Despite safety concerns that CCR5/CXCR4 antagonists are immune suppressors (2006, Treatment Update. 18: 5-6; 2006, Proj Inf Perspect. 7-12; 2006, AIDS Alert. 21: 11-2; 2006, AIDS Patient Care STDS. 20: 380; and 2006, AIDS Read. 16: 448) and evidence that HIV-1 can adapt to evade therapeutic agents that block the ECL2 domain of the CCR5 (Aarons et al., 2001, Virology. 287: 382-90), Human Genome Sciences began human clinical trials with an anti-CCR5 monoclonal antibody (2004, IAVI Rep. 9: 15). Other CCR5 inhibitors (2004, AIDS Alert. 19: 121, 123-4) showed promise and are being taken to clinical trial (2005 AIDS Patient Care STDS. 19: 59 and Jones et al., 2007, Eur J Med. Res. 12: 391-6). The HIV-1 co-receptors are a particularly attractive drug target because they have multiple transmembrane domains and a G-protein domain upon which small drugs can act (Leonard et al., 2006, Curr Med Chem. 13: 911-34). In addition, small molecule antagonists can be designed to bind both CCR5 and CXCR4, the two receptors that are known to be co-receptors for HIV-1 entry into lymphocytes (Ji et al., 2006, J Biomol Screen. 11: 65-74; Liu et al., 2007, Curr Pharm Des. 13: 143-62; Perez-Nueno et al., 2008, J Chem Inf Model. 48: 509-33; Rusconi et al., 2007, Cuff Top Med Chem. 7: 1273-89; and Wang et al., 2008, J Mol Graph Model. 26: 1287-95). Other drugs target the gp120-binding site on the virus or the cell surface protein disulfide isomerase (Ryser et al., 2005, 10: 1085-94). CCR5 has also been a popular target of vaccines, even though immune attack of CCR5 may result in decreased immunity. In 2007, the FDA approved Maraviroc, an imidazopyridine ligand that blocks CCR5 and the first receptor antagonist therapy for patients in whom multi-drug antiretroviral therapy have failed. Two double-blinded placebo-controlled trials included 1076 patients infected by HIV strains that used CCR5 co-receptor for entry into CD4+ lymphocytes. After 8 weeks, Maraviroc-treated patients had less viral burden, i.e. undetectable in 45.5% vs. 16.7% of placebo control, and over twice as many CD4+ cells. Maraviroc and other new antiviral drugs have adverse cutaneous reactions (Borras-Blasco et al., 2008, J Antimicrob Chemother. 62: 879-88). Other similar drugs are in phase 3 trials (Emmelkamp et al., 2007, Eur J Med Res. 12: 409-17). In summary, blockers of CCR5 not only prevent HIV-1 entry but also suppress viral replication. These include natural ligands of CCR5, including the MIP-1beta, MIP-1alpha, and RANTES. Antibodies against CCR5 also suppress HIV-1 infections. Several drugs bind CCR5 and CXCR4, as well as other HIV-1 binding sites.

### Umbilical Cord Blood (UCB) Therapy of AIDS

AIDS is associated with lymphopenia, particularly CD4+ lymphocytes, as well as shortage of naïve CD8+ T cells and non-lymphoid monocytes. UCB therapy may be beneficial for AIDS for several reasons. First, UCB should directly replenish t-cell populations and enhance immune function. Second, UCB may engraft and add to the stem cell population. Third, UCB blood cells tend to be more resistant to HIV infection than peripheral blood cells. UCB lymphocytes express CCR7 and CXCR4 while adult lymphocytes express more CCR5 (Loria et al., 2005, Cell Immunol. 236: 105-9). Fetal or neonatal lymphocytes are thus less susceptible to HIV-1 infection (Vicenzi et al., 2002, J Leukoc Biol. 72: 913-20). Chemokines that attract UCB lymphocytes to an injury site, i.e. MCP-1 and MIP-1alpha, downregulate their CCR5 expression (Jiang et al., 2008, Curr Neurovasc Res. 5: 118-24). Finally, monocyte-derived dendritic cells in cord blood (Folcik et al., 2001, J Hematother Stem Cell Res. 10: 609-20) have limited susceptibility to HIV infection due to lower expression of CD4 and CCR5, related to lower MIP1-alpha and MIP1-beta levels (Wang et al., 1999, J Acquir Immune Defic Syndr. 21: 179-88). UCB CD34+ cells express CCR1 and almost no CCR5 receptors (de Wynter et al., 1998, Stem Cells. 16: 349-56). Because HIV-1 does not bind CCR1, cord blood CD34+ cells are resistant to HIV-1 (Majka et al., 2000, Exp Hematol. 28: 1334-42) but, as they differentiate and express CCR5 (Hariharan et al., 1999, AIDS Res Hum Retroviruses. 15: 1545-52), their progeny may become susceptible to HIV (Zhao et al., 1998, J Infect Dis. 178: 1623-34). HIV-1 infection of CD34 cells and their progeny depends on membrane expression of CD4 receptor, as well as certain chemokine co-receptors. UCB CD4+ T cells are more immature than their adult counterparts (Delespesse et al., 1998, Vaccine. 16: 1415-9). Their activation depends on a CD28-mediated cosignal that dictate their cytokine profile and response to IL-2. CD28 activation of UCB cells lead to Th1 phenotype with increased IL1, IFN-gamma, and TNF-beta expression. In the absence of CD28 stimulation, the cells respond to IL-12 by producing IL-4 and IFN-gamma. CD8+ cells strictly require exogenous IL-4 to develop into IL4/5 producers. Subpopulations of UCB cells, however, are susceptible to HIV-1. For example, HIV-1 infects mast cells in cord blood and these cells may serve as a persistent HIV reservoir (Bannert et al., 2001, J Virol. 75: 10808-14). Occasional uncommitted hematopoietic cells may express CCR5 (Rosu-Myles et al., 2000, Stem Cells. 18: 374-81) and particularly CXCR4 (Loria et al., 2005, Cell Immunol. 236: 105-9). CD8+ T cells can be productively infected in vitro by macrophage tropic (M-trophic) HIV-1 isolates but are resistant to T cell-tropic (T-tropic) HIV strains (Yang et al., 1998, J Exp Med. 187: 1139-44). Activated UCB CD8 cells express high levels of CD4, CCR5, and CXCR4 and are susceptible HIV-1 infection. CD16+ cells in cord blood express high levels of CCR5 and are susceptible to HIV-1 (Jaworowski et al., 2007, J Infect Dis. 196: 38-42). Finally, T cells released from thymus in neonates have elevated CXCR4 expression (Berkowitz et al., 1998, J Immunol. 161: 3702-10), explaining why HIV-1 infected neonates develop high viremia levels and AIDS progresses rapidly (Sundaravaradan et al., 2006, Proc Natl Acad Sci USA. 103: 11701-6).

In summary, while most UCB cells monocytes, including CD34+ cells and CD8+ cells, tend to be resistant to HIV infections. HIV-1 can infect subpopulations of UCB cells, including mast cells. Immature CD8+ lymphocytes are resistant to HIV-1 but these cells express CD4, CXCR4, and CCR5 when they become activated. Thus, while UCB transfusions may benefit people with AIDS by replenishing their t-cells, engrafting and producing immune cells, the cells serve as targets for HIV-1 infection.

Non-Genetic Prevention of HIV-1 Infections of UCB cells

Several therapies can render UCB cells temporarily resistant to HIV-1. For example, chemokines that bind CCR5 can prevent HIV-1 infection of cord blood cells. In 1998, Chalita-Eid, et al. (Challita-Eid et al., 1998, AIDS Res Hum Retroviruses. 14: 1617-24) showed that a RANTES-IgG3 fusion protein is a potent inhibitor of HIV-1 infection of neonatal blood cells. Interferon-beta (IFN-beta) increases HIV-1 resistance of macrophages derived from cord blood CD34+ cells (Cremer et al., 2000, J Immunol. 164: 1582-7); correlating with upregulation of RANTES and reduced CCR5 expression. Interferon gamma (IFNgamma) upregulates CCR5 expression in cord blood phagocytes (Hariharan et al., 1999, Blood. 93: 1137-44), it reduces CD4 expression and inhibits HIV replication in cord blood monocytes (Creery et al., 2004, Clin Exp Immunol. 137: 156-65) by elevating expression of SDF-1 and RANTES. Beta chemokines block HIV-1 replication (Ketas et al., 2003, AIDS Res Hum Retroviruses. 19: 177-86). Auto-immunity to CCR5 can protect UCB cells against HIV-1 infections. In 2008, Lobo, et al. (Lobo et al., 2008, J Immunol. 180: 1780-91) reported that IgM antileukocyte autoantibodies naturally bind CD3, CD4, CCR5, and CXCR4, inhibit T-cell activation and chemotaxis, and protect cells against HIV-1 infections of (Lobo et al., 2008, J Immunol. 180: 1780-91). Ditzel, et al. (Ditzel et al., 1998, Proc Natl Acad Sci USA. 95: 5241-5) found that CCR5 receptor acts as an alloantigen in CCR5Δ32 homozygous individuals and auto-antibodies from the serum of such individuals competed for radiolabeled RANTES binding to the CCR5 receptor.

These autoantibodies may be useful for selecting CCR5+ UCB cells. Vaccine induced auto-antibodies against CCR5 can also reduce infection HIV-1 infection rates of macque monkeys. Some investigators have engineered RANTES to enhance antiviral activity of the molecule, while reducing or abrogating its inflammatory properties (Vangelista et al., 2008, Vaccine. 26: 3008-15). For example, Sun, et al. (Sun et al., 2008, J Virol Methods) used the CCR5 ligand RANTES combined with a endoplasmic reticulum sequence (RANTES-KDEL) that retained the molecule on endocytoplasmic reticulum to trap the CCR5 receptor protein and reduce surface expression of the receptor. A similar approach presumably could be used with ligands for the other co-receptors. In 2002, the discovery of RNA interference suggested the possibility of blocking CCR5 expression to confer resistance to AIDS. An, et al. (An et al., 2007 Proc Natl Acad Sci USA. 104: 13110-5) demonstrated stable expression of siRNA that inhibits CCR5 expression by CD34+ hematopoietic stem/progenitor cell transplants. Anderson, et al. (Anderson et al., 2007, Mol Ther. 15: 1182-8) developed a lentiviral vector containing three anti-HIV genes (CCR5 ribozyme, tat-rev siRNA, and TAR decoy) in SCID-hu mouse-derived T-cells. Injected into SCID mice, these transfected cells produce T cells that are relatively resistant to HIV-1 infection. Leukemia inhibitor factor (LIF) inhibits HIV-1 replication via restriction of stat 3 activation. Tjernlund, et al. showed that LIF markedly inhibited HIV-1 repication in vitro and in human organ explant cultures. LIF activates the Jak/Stat signaling pathway. Pretreatment of cells with recombinant human LIF significantly reduced uptake of HIV-1 viral particles. Likewise, HIV-1 replication can be restricted by TRIM5alpha siRNA (Pineda et al., 2007, Virology. 363: 310-8).

Genetic Methods of Preventing HIV-1 Infection of UCB cells

The prevalence of CCR5Δ32 allele in umbilical cord blood from Caucasian Europeans may be as high as 10%. The prevalence appears to be rising since studies of the CCR5Δ32 allele suggest that it was only present in 5% of DNA samples from medieval Poland whereas current day estimates are 10.26% (Zawicki et al., 2008, Infect Genet Evol. 8: 146-51). Presumably, the prevalence of the gene has been rising because the selective advantage that it gives to carriers of the gene over individuals that don't have the gene. One approach is to collect umbilical cord blood units that possess the CCR5Δ32 allele and expand these units so that they can be used to treat many more people. The CKR5Δ32 mutation is not the only source of genetic resistance to HIV infection. African infants that express multiple gene copies of CCL3 (MIP1-alpha) are less susceptible to HIV infection (Kuhn et al., 2007, Aids. 21: 1753-61). Copy number of CCL3L1 correlates with decreased susceptibility to HIV-1 (Bugeja et al., 2004, Aids. 18: 1069-71 and Gonzalez et al., 2005, Science. 307: 1434-40). An analysis of peripheral blood leukocytes from uninfected infants born to HIV-1 infected mothers indicate that uninfected babies had high proportions of CXCR4-expressing cells and few CCR5-expressing cells (Shalekoff et al., 2004, Clin Diagn Lab Immunol. 11: 229-34). Variations in genes encoding CCLL1-CCR5 genotypes are associated with altered cell mediated immunity to HIV-AIDS (Dolan et al., 2007, Nat Immunol. 8: 1324-36). Other genes that affect HIV susceptibility (Arenzana-Seisdedos et al., 2006, Semin Immunol. 18: 387-403) include CCR2, CX3CR1, MIP-1alpha, MIP-1beta/CCL4, RANTES/CCL5 and SDF-1/CXCL12 genes. Yoshida, et al. (Yoshida et al., 2008, Traffic. 9: 540-58) showed that an N-terminal deletion of CD63 (i.e. CD63DeltaN) blocks HIV-1 entry by suppressing CXCR4 surface expression. Deletion or knockout of the CCR5 gene may have undesirable side effects. The CCR5 chemokine receptor regulates chemotaxis of leukocytes and play an important role in immunological processes (Tian et al., 2008, Cell Signal. 20: 1179-89), as well angiogenesis (Wu et al., 2008. J Immunol. 181: 6384-93). Deletion or mutation of CCR5 may affect ability of cord blood cells to carry out immune function. For example, the CCL3L1-CCR5 genotype influences durability of immune recovery during antiretroviral therapy of HIV-1 infected individuals. Changes of CCR5 also may increase risk of autoimmune diseases. For example, polymorphisms of CCR5 are associated with autoimmune diseases such as systemic lupus erythaematosus (Mamtani et al., 2008, Ann Rheum Dis. 67: 1076-83). Finally, some HIV-1 viruses don't use CCR5 to enter cells and blocking CCR5 expression does not provide complete protection. Genotypic algorithms are available to determine HIV-1 tropism, to predict success of co-receptor antagonism (Soulie et al., 2008, HIV Med. 9: 1-5). The CKR5Δ32 or CCR5Δ32 mutation may do more than act as a dominant negative. It was found that the CCR5 receptor protein must be processed by endoplasmic reticulum and be phosphorylated and multimerized before surface expression (Benkirane et al., 1997, J Biol Chem. 272: 30603-6) The mutant CCR5Δ32 can form complexes with CCR5 but cannot be phosphorylated. Without phosphorylation, the heterocomplex cannot be expressed on the cell surface, thereby reducing CCR5 expression more than expected from simple heterozygous expression of a non-working receptor protein. However, much more efficient and effective methods of suppressing CCR5 expression have became available and will be described below.

### Gene Suppression Methods

Many methods are available for suppressing CCR5 expression. In 2000, Cagnon & Rossi (Cagnon et al., 2000, Antisense Nucleic Acid Drug Dev. 10: 251-61) developed a hammerhead ribozyme that targets CCR5 mRNA and down-regulates CCR5 expression in cells, using an adenovirus polymerase to express the transcript in cells and showing that this down-regulated CCR5 expression by 70%. In 2000, Bai, et al. (Bai et al., 2000, Mol Ther. 1: 244-54) used retrovirus to transfect anti-CCR5 ribozyme (R5Rbz) into CD34+ cells and showed that macrophages differentiated from these transfected cells resist HIV-1 infection. Bai, et al. (Bai et al., 2001, AIDS Res Hum Retroviruses. 17: 385-99) subsequently constructed an anti-CCR5 ribozyme heterotrimer that targets three cleavage sites in CCR5 mRNA, showing that this inhibited CCR5 surface expression and reduced HIV-1 infection by 70%. However, many of these methods were superseded by RNA interference (RNAi). HIV-1 specific RNAi therapy, i.e. short-inhibiting RNA (siRNA) and short-hairpin RNA (shRNA), are very efficient ways of reducing CCR5 (Boden et al., 2004, Curr Opin Mol Ther. 6: 373-80) and CXCR4 (Zhou et al., 2004, Gene Ther. 11: 1703-12) expression. In 2002, Martinez, et al. (Martinez et al., 2002, Aids. 16: 2385-90) showed that siRNA that target CXCR4 and CCR5 selectively stopped cell surface expression of these co-receptors without affecting each other or CD4 expression. Novina, et al. (Novina et al., 2002, Nat Med. 8: 681-6) reported successful suppression of CD4 expression, the viral structural Gag protein, or Nef regulatory protein. Anderson & Akkina (Anderson et al., 2007, Mol Ther. 15: 1182-8) showed that lentiviral vector-expressed siRNA knocked down CCR5 and protected transgenic macrophages against HIV-1 infection. Anderson, et al. (Anderson et al., 2003, Oligonucleotides. 13: 303-12) used bispecific siRNA that targets CD4, CXCR4, and CCR5. Tamhane and Akkina (Tamhane et al., 2008, AIDS Res Ther. 5: 16) used the Sleeping Beauty transposon system to transfer CCR5 and CXCR4 siRNA, red fluorescent protein (RFP) reporter, and a drug-selectable neomycin resistance gene, using a hyperactive transposase (HSB5) to transfer the plasmids into cells expressing CD4, CCR5, and CXCR4. This shut down CCR5 and CXCR4 surface expression in the cells. Kumar, et al. (Kumar et al., 2008, Cell. 134: 577-86) used T-cell specific siRNA against CCR5, showing that anti-CCR5 and antiviral siRNAs complexed to T-cell CD7-specific single chain antibody conjugated to the oligo-9-arginine peptide (scfvCD7-9R) can be specific for T-cells, controlled viral replication, and prevented disease-associated CD4 T cell loss. Bhattacharyya, et al. (Bhattacharyya et al., 2008, Scand J Immunol. 67: 345-53) found that CCR5-specific siRNA reduced parasitic burden of Leishmaniasis in murine macrophages by 70%. Poluri & Sutton (Poluri et al., 2008, Mol Ther. 16: 378-86) showed that gene transfer vectors encoding short hairpin RNA (shRNA) against CCR5 reduced viral titers in cells by >30-fold. RNA interference can be directed at genes besides CCR5 or CXCR4. Lim, et al. (Lim et al., 2008, Mol Ther. 16: 565-70) used siRNA against the 5′-long-terminal repeat (5′LTR) promoter of HIV-1 and suppressed productive infection of 2 different cell lines expressing CD4, CCR5, and CXCR4. Harmon & Ratner (Harmon et al., 2008, J Virol. and Harmon et al., 2008, J Virol. 82: 9191-205) showed that induction of the Galpha (q) signaling cascade is necessary for viral entry and Galpha inhibitors or siRNA will block viral entry. Chen, et al. (Chen et al., 2008, Virology. 379: 191-6) showed that CD63 plays a critical role in HIV replication and infection of macrophages and cell lines and that siRNA against CD63 will prevent both. Finally, Tian, et al. (Tian et al., 2008, Cell Signal. 20: 1179-89) targeted siRNA against hematopoietic-specific G(16) and G(14), which link the G(i)-coupled receptors CCR1, CCR2a, CCR2b, CCR3, CCR5, and CCR7. This could reduce the expression of multiple receptors. Finally, the siRNA can be directed against CCR5 promoter (Giri et al., 2005, Am J Physiol Cell Physiol. 289: C264-76).

### Combinatorial Anti-Viral Gene Therapy

Combinatorial gene therapies target multiple mechanisms of HIV-1 entry and replication. In 2003, Akkina, et al. (Akkina et al., 2003, Anticancer Res. 23: 1997-2005) proposed using siRNA against viral envelope proteins tat and rev, anti-CCR5 ribozymes, and RNA (TAR) decoys together. RNAi directed at viral envelope RNA, such as rev and tat, suppress viral reproduction (Akkina et al., 2003, Anticancer Res. 23: 1997-2005). The TAR decoy aptamer is a nucleolar localizing decoy that binds and sequesters the HIV Tat protein but does not interfere with normal thymopoiesis (Banerjea et al., 2004, AIDS Res Ther. 1: 2). Using lentiviral vectors expressing PolIII-promoted anti-HIV RNA and anti-CCR5 ribozymes, Li, et al. (Li et al., 2003, Mol Ther. 8: 196-206) showed that this combination efficiently protected against HIV-1 infection.

In 2004, Banerjea, et al. (Banerjea et al., 2004, AIDS Res Ther. 1: 2) used lentiviral transduction of TAR Decoy and CCR5 ribozyme into CD34+ progenitor cells to create HIV-1 resistant T cells and acrophages. In 2006, Li, et al. (Li et al., 2006, Ann NY Acad Sci. 1082: 172-9) used multiple RNAi in combination with a CCR5 ribozyme and TAR decoy to treat HIV infection of hematopoietic cells. In 2007, Anderson, et al. (Anderson et al., 2007, Mol Ther. 15: 1182-8) used a lentiviral vector containing three anti-HIV genes or triple-R (anti-CCR5 ribozyme, tat-rev siRNA, and TAR decoy) to produce phenotypically normal T cells that effectively resist HIV-1 infection. Morris, et al. (Morris et al., 2005, RNA Biol. 2: 17-20) tested multiple siRNA targeting HIV-1 gag, vif, tat, rev, and host CD4 and CCR5, finding that sequence divergence of HIV-1 strains severely limit the use of anti-viral siRNA. The lentiviral vector is popular for tranducing genes into cells because it infects nondividing cells with high efficiency and can deliver multiple genes (Banerjea et al., 2004, AIDS Res Ther. 1: 2). Qin, et al. (Qin et al., 2003, Proc Natl Acad Sci USA. 100: 183-8) showed that lentivirus can routinely transfect over 40% of peripheral T-lymphocytes with CCR5 siRNA that reduces CCR5 expression by over tenfold and reduces the number of infected cells by 3-7 fold. Song, et al. (Song et al., 2003, J Virol. 77: 7174-81) showed gene silencing from siRNA was sustained for over 15 days in non-dividing cells, such as macrophages.

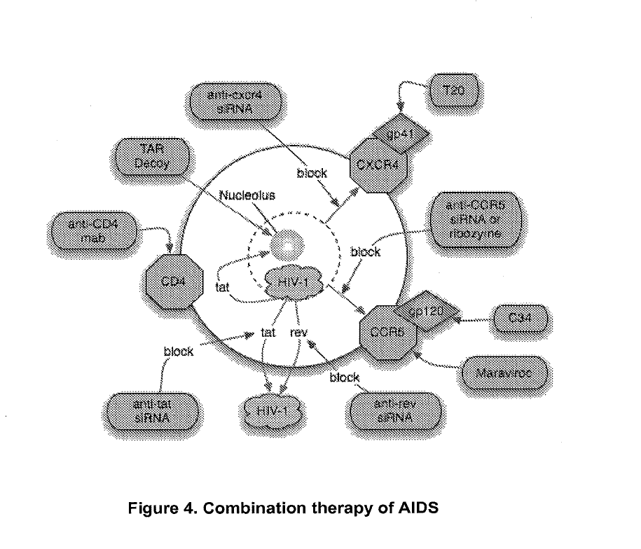

The development of drug combinations that target the HIV reverse transcriptase and protease enzymes revolutionized the treatment of HIV/AIDS but problems with these agents, such as viral escape mutants (Ray et al., 2007, J Virol. 81: 3240-50 and Shafer et al., 2008, AIDS Rev. 10: 67-84), persistent viral reservoirs, compliance with complicated drug regimens, and toxic side-effects have limited the usefulness of these drugs for some patients. Aside from CD4, CCR5 and CXCR4 receptor blockade, one important class of anti-HIV treatment blocks the action of the fusogenic envelope glycoprotein gp120 (Liu et al., 2008, J Mol Model. 14: 857-70; Platt et al., 2007, J Mol Biol. 374: 64-79; and Shafer et al., 2008, AIDS Rev. 10: 67-84) and gp41 (Jacobs et al., 2008, Vaccine. 26: 3026-35; Sougrat et al., 2007, PLoS Pathog. 3: e63; and Zahn et al., 2008, Gene Ther. 15: 1210-22), including the fusion inhibitor T20 (enfurvirtide) which is useful for preventing HIV-1 infections of macrophages (Yi et al., 2008, J Acquir Immune Defic Syndr. 47: 285-92) and C34 that blocks HIV infection of langerhans cells and T-cells. As shown in FIG. 4, multiple drugs can be used to interfere with viral infection and reproduction in the cells. These include drugs that block receptors (CD4, CXCR4, CCR5), fusogenic glycoproteins (gp41, gp120), viral envelope proteins (tat, rev), and ribozymes and siRNA that block the production of CCR5 and CXCR4 receptors on cell membranes. Of these drugs, the ones that prevent viral entry are probably the best. Once the cells have entered the cell, preventing viral replication may slow down spread of the infection but does not prevent the cells becoming reservoir for the viruses. However, it is probably important to treat the patients with combination anti-virals before engraftment.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### Materials and Methods

We propose the following approach to produce HIV-1 resistant umbilical cord blood cells, verifying their ability to engraft in immune-deficient mice, and then assessed in clinical trial of children and adults with AIDS.

Transfection. The approach utilizes the Amaxa (http://www.amaxa.com) nuclear targetting electroporation method with superfect and lipofectamine cationic lipid plasmids (http://www.invitrogen.com) to insert the following genes into umbilical cord blood mononuclear cells: siRNAs against CCR5 and CXCR5, GFP (green fluorescent protein), and the PGK (phosphoglycerate kinase) neomycin resistance gene. The GFP serves as a marker of successful gene transfer while the neomycin gene allows us to select transfected cells in the presence of lithium which stimulate proliferation of cord blood mononuclear cells (CBMC).

Verification. After transfecting the cells, it is verified that the transfected CBMC cells engraft and do not express CCR5 and CXCR5 co-receptors for several generations. The cells are then transplanted into immune-deficient mice to demonstrate that the transfected cells engraft into bone marrow and produce blood cells, including neutrophils and lymphocytes. After that, the cells are tested for their resistance to HIV-1 infections in vitro. Once confirmed, the following clinical trials are then conducted.

Clinical trial of autologous cord blood transfusion. Umbilical cord blood samples are collected from children that are born of HIV-1 infected mothers. If these children show evidence of HIV infection, mononuclear cells are also isolated from the cord blood, and transfect the cells in the above manner, and then transfuse the modified cells into the child. At various times after transplantation, blood samples are collected to determine whether engraftment has occurred (i.e. presence of blood cells that express green fluorescent protein). The viral load is then determined, particularly in transplanted GFP expressing cells. The primary endpoint is engraftment, production of HIV-1 resistant immune cells, and restoration of immune function. The secondary endpoint is the time-course of AID in the subjects.

Clinical trial of heterologous cord blood transfusions. In babies that do not have umbilical cord blood collected, we transfect units of HLA-matched cord blood with siRNA against CCR5 and CXCR4, and transplant these cells into babies with HIV-1 infections. Again, the primary endpoint of the trial is whether the transfected cells have engrafted and are producing HIV-1 resistant cells without graft-versus-host-disease. The secondary endpoint is the time-course of AIDS in the subjects. It is expected that the symptoms of AIDS will decline as the more and more cells are produced by the transplanted hematopoietic cells. It is possible that there will be a resurgence of AIDS as successive generations of cells produce less siRNA against CCR5 and CXCR4. Each of the above will be discussed in greater detail below.

### EXAMPLE 1

### Transfection

A non-viral method is used to transfect and over express four genes into human cord blood mononuclear cells: the siRNAs for CCR5 and CXCR4, GFP, and the neomycin resistance gene. After transfecting the cells, the transfection rate is verified by the percentage of cells expressing GFP and use the neomycin resistance genes to select transfected cells. The resulting cells should all be expressing GFP but not CCR5 or CXCR4 of their surfaces (by immunhistochemistry).

Rationale. The CCR5 is the main receptor for HIV-1 to enter lymphocytes while CXCR4 siRNA should reduce or prevent expression of CXCR4 on monocytes and macrophages. The GFP gene express GFP protein and allows the cells to be detected. The neomycin resistance gene allows us to use neomycin to select and purify the transfected cells. Use of a non-viral electroporation method of transfecting the cells minimize the burden of proving the safety of the cells. In our experience, the Amaxa electroporation method has been very efficient, allowing transfection of over 80% of cells with the GFP gene. Using a non-viral approach to transfecting the cells should increase the safety and the burden for demonstrating the safety of the cells. Because the electroporation method does not make permanent changes of the genomes, the likelihood of neoplasms or other problems is low. Likewise, because the cells stop expressing CCR5 and CXCR4 only for several generations, it should not compromise their immune or stem cell function for long but long enough to allow protection against HIV-1.

Expected results: We may have to introduce other genes to suppress CCR5 and CXCR4 surface expression completely and for a longer period of time. To do the latter, we may have to go to a retroviral or lentivirus approach to insert the genes into the genome. It is expected that the cells are transfected and express the GFP.

### EXAMPLE 2

### Verification

In this example, assays are conducted to verify that The transfected cells engraft in immune-deficient animals and continue to have little or no expression of CCR5 and CXCR4 for several generations. Assays are also conducted to engraft human CBMC into immune-deficient mice (NOD/SCID/IL2Rgamma null mice) that have been irradiated to damage their bone marrow. The goal of the experiments is to show the cells engraft and produce HIV-resistant cells.

Rationale. The goal of these experiments is to show that the transfected cells are still able to function as hematopoietic cells, producing immune cells. It is expected that the transfecting cells form CFUs (colony forming units). To do so, we transplant the human cells into immune-deficient mice. These mice normally accept human cord blood mononuclear cell transplants without myeloablation (Watanabe et al., 2007, Blood. 109: 212-8).

Note that monkey experiments may not be helpful in this situation. For example, in order to get human cells to engraft in monkeys, we will have to use immunosuppression, such as cyclosporin or FK506, which would interfere with the engraftment of the cells and the immune function of cells produced by the engrafted cells (Gardner et al., 1998, Exp Hematol. 26: 991-9).

Expected Results: It is expected that the transfected cells are engrafted in immune-suppressed animals.

### EXAMPLE 3

### Clinical Trial of Autologous Cord Blood Transplant

The first group of patients are tested are children born of HIV-infected mothers, particularly those with high viral loads. Cord blood are collected at the time of birth. If the child develops evidence of HIV-infection, the unit of cord blood cells are transfected with siRNA against CCR5 and CXCR4, GFP, and neomycin resistance green.

Rationale. Although HIV does not usually pass through the placental barrier (Rogers et al., 1986, Obstet Gynecol. 68: 2S-6S) and only 2.7-4.0% of umbilical cord blood from HIV-infected mothers are seropositive (Lester et al., 1992, West J Med. 156: 371-5; Nicholas et al., 1994, Arch Pediatr Adolesc Med. 148: 813-9; and Sperling et al., 1989, Obstet Gynecol. 73: 179-81) and the rest are sero-negative, children of HIV-infected mothers with have a high risk of becoming infected (Pedersen et al., 2007, PLoS ONE. 2: e838), especially when mother has a heavy viral load. Cord blood lymphocytes are not especially sensitive to HIV-1 (Krogstad et al., 1994, AIDS Res Hum Retroviruses. 10: 143-7) and mothers that are treated with anti-virals can avoid passing the disease to their babies (Ripamonti et al., 2007, Aids. 21: 2409-15). Nevertheless, the umbilical cord blood of these children should be useful for treating those who become infected after birth. Because the cord blood is autologous, they should match. We would transfect the cells with CCR5 and CXCR4 siRNA, GFP, and neomycin resistance gene and then transfuse the blood back into the child, follow the child to see if the cells engraft and what effect the transfusion has on their immune function and the course of the AIDS. The trial will tell us whether the transplanted cells engraft and are immune to HIV.

Expected Outcome: We expect to get the cord blood to engraft and produce several generations of HIV-immune cells. Combined with anti-viral therapies, this may lead to a cure of some of the children. We also expect to find a temporary improvement and a resurgence of the virus in some children. The risk is low and the benefit is potentially substantial.

### EXAMPLE 4

### Clinical Trial in Heterologous HLA-Matched Cord Blood Transplant

We then determine whether CRR5 and CXCR4 siRNA transfected units of HLA-matched cord blood can be transplanted to HIV-infected children and whether they are beneficial. The trial focuses on children with HIV-1 infections that have become refractory to combination anti-viral drugs and are showing evidence of immune-compromise.

Rationale. If CCR5 and CXCR4 siRNA transfected heterologous HLA-matched cord blood units are resistant to HIV-1 and improve the immune status of HIV-1 infected children, this would expand the therapeutic approach to children that did not have cord blood collected at birth. Most of these presumably would be older children and are on the verge of failing their anti-viral therapies These patients have few other drugs to go to. Their immune system should be failing and engraftment of HLA-matched cord blood should improve their immune function. The primary outcome measure is the appearance of the progeny of the engrafted cells. If there is any HLA mismatch, this can be used to identify the grafted cells from the host.

Expected Outcome: Treatment with HIV-1 resistant cord blood, especially in patients who may CNS symptoms, is not likely to eliminate the virus from all potential reservoirs. On the other hand, the treatment should restore the immune system to some extent and therefore benefit the patient. The treatment may be more effective in an individual who is still relatively early after HIV infection and has not has a chance to have HIV infections in many other places. In 1995, Ho, et al. (Ho et al., 1995, Stem Cells. 13 Suppl 3: 100-5) demonstrated a highly efficient transduction of CD34+ cells from placental and umbilical cord blood by retrovirus bearing the ribozyme gene which rendered monocytes resistant to HIV-1 infection. The ribozyme is not as effective as siRNA or the CCR5 gene. Battacharya, et al. (Bhattacharya, 2006, Clin Exp Obstet Gynecol. 33: 117-21) treated 123 HIV-positive patients who have anemia and emaciation with fresh umbilical cord blood, finding that the transfusion significantly reduced fatigue and improved the energy level of the patients, as well as a sense of well-being and weight gain. Because this is without matching, these beneficial effects presumably are direct effects of the cord blood cells.

### DISCUSSION

We propose to conduct the following studies in three stages. In the first stage, we focus on inserting the CCR5 and CXCR4 siRNA, GFP and NRG into neonatal mouse and blood mononuclear cells. Our goal is to produce HIV-resistant cells that engraft and produce colony forming hematopoietic cells.

In the second stages, we study the effects of the cells in immune-deficient rats and mice, to see if the genetically modified cells engraft and produce immune cells and restore immune function in the animals. In the third stage, we carry out two clinical trials. One trial focuses on autologous cord blood units collected at birth, treated to be HIV-resistant, and transfuse the units back to babies of HIV-infected mothers. The other assess heterologous cord blood units that are genetically modified to resist HIV infection. Umbilical cord blood has been successfully used to treat people with a wide variety of hematopoietic disorders, including leukemia, anemia, auto-immune, and immune deficiency syndromes. By restoring the immune function, cord blood cells should be beneficial to patients with AIDS. However, HIV-1 will infect the transplanted cells unless something is done to immunize them against HIV. In the past decade, the CCR5 and CXCR4 receptors have been shown to be co-receptors necessary for most HIV-1 viruses to enter cells. Many investigators have shown that blockade of these co-receptors by a variety of methods, including preventing surface expression of the co-receptors, can make cells resistant to HIV-1 infections.

We use the following method to increase resistance of cord blood cells to HIV-1 infections. We isolate mononuclear cells from the cord blood units, using the Ficoll gradient with DNAase, and then use electroporation (Amaxa) to introduce siRNA to block CCR5 and CXCR4 genes in the mononuclear cells. In addition, we include a green fluorescent TV and as well as a neomycin resistance gene (NRG) to identify and purify cells that have been successfully transfected. After verifying that the transfected cells are HIV-1 resistant and produce colony-forming hematopoietic units, we ascertain whether the cells engraft and restore immune function in immune-deficient animals, and then test these HIV-1 resistant cells in clinical trials of babies born of mothers with HIV infections.

The clinical trials focus on the primary endpoint of hematopoiesis of engrafted GFP expressing immune cells and a secondary endpoint of reducing AIDS symptoms and reducing viral burdens in HIV-1 infected babies. If the cells engraft and produce cells that are resistant to HIV-1 infection, this should correct the immune deficiency and reduce the population of infected cells. In babies, we test initially autologous cord blood and then heterologous HLA-matched blood. The results are expected to show that both autologous and HLA-matched heterologous cells engraft, produce hematopoietic cells, and does not cause serious graft-versus-host-diseases. Finally, trials will establish the feasibility of the method and determine the efficacy of engrafting autologous and HLA-matched umbilical cord blood cells to treat babies and adults with AIDS.

Alternative Gene Suppression Approaches

Electroporation to introduce the CCR5 and the CXCR4 siRNA genes may not produce sufficient expression of the siRNA to eradicate the HIV-1 infection because the genes may not be carried over into many generations. On the other hand, this non-viral method of transiently suppressing CCR5 and CXCR4 expression in the cells may be effective in reducing HIV-1 infection and improving immune function of the patients. Permanent suppression of both of these crucial chemokine receptors may also have a deleterious effect the immune function of the cells.

A recent study by Tamhane, et al. (Tamhane et al., 2008, AIDS Res Ther. 5: 16) reported successful use of the non-viral Sleeping Beauty Transposon system for introducing CCR5 and CXCR4 siRNA. Although lentiviral vectors have been successfully used to transpose CCR5 and CXCR4 siRNA genes, the SBT system produced stable gene transfer of CCR5 and CXCR4 siRNA, resulting in marked viral resistance of MAGI-CCR5 and MAGI-CXR4 cell lines. This approach is attractive because it doesn't use viruses insert the gene. If the above does not work, a retrovirus or lentivirus system can be used for the same purpose. Many investigators have used these viruses for genetic modification and introduction of siRNA's to cells.

Clinical Trial Considerations

Engraftment of the cells and satisfactory hematopoiesis is assessed from the presence of GFP-expressing cells in blood and restoration of immune function in immune-deficient mice and then in humans. Note that proof of concept can be achieved with several subjects. Demonstration of safety, however, will take more subjects. We therefore plan to test approximately 10 patients in the autologous and 10 patients in the heterologous transplant trials. If the cord blood cells engrafted and the patients recovered immune function and viral presence declined or disappeared, this would mean that the treatment is successful. We expect that engraftment occur without myeloablation.

The likelihood of graft-versus-host-disease (GVHD) is very low with autologous transplants. However, it may occur with the heterologous transplants. Should GVHD happen, we will of course treat the patients as we would normally with glucocorticoids and anti-inflammatory drugs. However, note that the presence of GVHD would suggest that the grafted cells are capable of immune responses. We would also be able to track the number of cells produced by the grafted cells if there were any mismatch of HLA. The first few generations of the cells should be GFP positive. The proposed therapy poses little or no risk to an infant that is already infected with HIV if no myeloablative chemotherapy is used, the cord blood is simply transfused, and subject blood tests are obtained from a central venous line that is used both for infusion of the cells as well as for sampling of blood before and after the treatment. Because the trial focuses on young babies and children, the dose of cells in a single cord blood units should be sufficient to produce satisfactory engraftment of the cells. The autologous blood transfusion, in particular, should pose little or no danger to the patient. Precautions will be taken in the handling and analysis of blood samples from AIDS patients in the trials. For testing the HIV-1 susceptibility of the genetically altered cells, we will send the cells to laboratories that are equipped to handle HIV infections.

Likewise, all samples from patients with AIDS are analyzed in facilities that are suitably equipped to handle HIV-1 infected samples. Other than the blood tests, contact with an infectious material will be strictly limited. For example, the processing of the cord blood from babies born of HIV-infected mothers will have to be done under special facilities equipped for HIV-1 studies.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a human subject having, or at risk of having, an HIV infection, the method comprising obtaining human umbilical cord blood cells containing a first RNAi agent that represses the expression of CCR5 and a second RNAi agent that represses the expression of CXCR4; administering to a human subject in need thereof an effective amount of the umbilical cord blood cells.

2. The method of claim 1, wherein the subject is a baby born of a mother that has an HIV infection.

3. The method of claim 1, wherein the umbilical cord blood cells are autologous to the subject.

4. The method of claim 1, wherein the umbilical cord blood cells are obtained by a process comprising transiently transferring into the cells (1) the first RNAi agent or a first nucleic acid encoding the first RNAi agent and (2) the second RNAi agent or a second nucleic acid encoding the second RNAi agent.

5. The method of claim 4, wherein the process further comprising introducing into the cells a recombinant nucleic acid encoding a selectable marker protein, and enriching the cells expressing the selectable marker protein.

6. The method of claim 1, wherein the umbilical cord blood cells further contain a third RNAi agent that repress the expression of a gene selected from the group consisting of CD4, HIV-1 gag, HIV-1 vif, HIV-1 tat, and HIV-1 rev.

7. An isolated human umbilical cord blood cell that contains a first RNAi agent that represses the expression of CCR5 and a second RNAi agent that represses the expression of CXCR4.

8-9. (canceled)
